SEQUENCE LISTING (2 sequences)

SEQ ID NO: 1 - protein, 1464 aa, unknown
  COL1A1
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 54 aa, unknown
  selected gelatin-like peptide with high Tg
GDRGETGPAGPPGAPGAPGAPGPVGPAGKSGDRGETGPAGPAGPVGPAGARGPA

(19) United States
(12) Patent Application Publication
(10) Pub. No.: US 2007/0031501 A1
(43) Pub. Date: Feb. 8, 2007

(54) Use of recombinant or synthetic gelatin-like proteins as stabiliser in lyophilized pharmaceutical compositions

(76) Inventors: Andries Van Es, Dorst (NL); Jan Bastiaan Bouwstra, Bilthoven (NL); Yuzo Toda, Goirle (NL)

Correspondence Address: ROGER PITT;KIRKPATRICK & LOCKHART NICHOLSON GRAHAM LLP, 599 LEXINGTON AVENUE, 33RD FLOOR, NEW YORK, NY, 10022-6030, US

(21) Appl. No.: 10/566,878

(22) PCT Filed: Aug. 4, 2004
(86) PCT No.: PCT/NL04/00552

§ 371 Date: Feb. 2, 2006

(30) Foreign Application Priority Data
Aug. 5, 2003 (EP) 03077451.7

(51) Int. Cl. A61K 9/14; C07K 14/78

(52) U.S. Cl. 424/488; 530/354

(57) ABSTRACT

The invention relates to the use of gelatin-like proteins, or polypeptides, with an increased calculated glass transition temperature as stabilisers in lyophilized biological or pharmaceutical compositions.

### FIELD OF THE INVENTION

The invention relates to the use of gelatin-like proteins—or polypeptides—as stabilisers in lyophilized biological or pharmaceutical compositions.

### BACKGROUND OF THE INVENTION

A well-established application of gelatin is the use as stabilizer for physiologically active substances in lyophilized biological or pharmaceutical compositions. Lyophilization or freeze drying of physiologically active substances is generally done in the presence of a stabiliser and a disaccharide. Freeze drying compositions and -processes are empirically determined for different types of physiologically active substances, as described by D. Greiff in Developments in Biological Standardization (1992), 7 Biol. Prod. Freeze Drying Formulation), 85-92. The stability of the lyophilized composition depends on several factors like the nature of the physiologically active substance, and water content and glass transition temperature (Tg) of the freeze-dried composition. Vaccines are examples of pharmaceutical compounds stored as freeze-dried compositions.

Vaccines are used amongst others in development countries where the sometimes severe storage conditions for vaccines can be difficult to maintain. Stability of lyophilized vaccines is a major concern, and the World Health Organisation issues strict rules for storage of such compositions.

Physiologically active substances are for example vaccines, (therapeutic) proteins, enzymes, (monoclonal) antibodies and the like. Gelatin is a preferred stabiliser because of its known low immunogenicity. Care should be taken that the gelatin solution is made sterile, pyrogen and antigen free.

A disadvantage of the presently used gelatin is the possibility of immediate hypersensitivity, which can occur upon application of the presently used gelatin derivatives, known as anaphylactic shock.

Another disadvantage of the commercially used gelatin derivatives is the fact that the gelatin used is isolated from animal sources such as animal bone and hide, in particular it is derived from bovine sources. Disadvantages of this material are the presence of impurities and the fact that the nature of the composition is not clearly defined and thus not reproducible. This may impose additional screening to ensure that the derivatisation process results in a product with the desired properties and may require careful purification steps. An additional problem nowadays, especially in relation to gelatin isolated from bovine sources, is the risk of contamination of the gelatin with factors responsible for the occurrence of Bovine Spongiform Encephalitis (BSE). For this reason the use of gelatin in pharmaceutical compositions may be prohibited.

WO 01/34801 A2 describes generally the use of recombinant gelatins as vaccine stabiliser to avoid the obvious problems associated with the use of natural gelatin. However, it is silent with respect to further advantages, which can be achieved by specifically designed recombinant structures.

EP 0,781,779 A2 describes the use of a gelatin of not more than 20 kiloDalton (kDa) that is hydrolyzed specifically by collagenase to render it non-antigenic. U.S. Pat. No. 4,147,772 describes the use of hydrolyzed gelatin of about 3 kDa as a nongelling matrix with little antigenicity.

U.S. Pat. No. 4,273,762 describes an attempt to reduce the lyophilization time of vaccines which have partly hydrolized gelatin as stabiliser.

### SUMMARY OF THE INVENTION

It is an object of the invention to provide improved stabilisers for lyophilized compositions comprising physiologically active substances.

It is also an object of the invention to provide lyophilized compositions comprising the improved stabilisers, said compositions having an improved stability.

It is a further object of the invention to reduce the lyophilizing time for compositions comprising physiologically active substances with the improved stabiliser.

Surprisingly it was found that these objectives were met by using as a stabilizer a recombinant or synthetic polypeptide comprising at least one stretch of 10 or more consecutive repeats of Gly-Xaa-Yaa triplets and in which at least 20% of the amino acids are present in the form of consecutive Gly-Xaa-Yaa triplets and said recombinant or synthetic polypeptide having a calculated glass transition temperature of higher than about 180 degrees Celsius, as calculated by formula 8 and 9 of Matveev as published in Food Hydrocolloids Vol. 11 no. 2 pp. 125-133, 1997. A peptide with these characteristics is hereinafter referred to as “recombinant” or “synthetic collagen-like peptide (or polypeptide)” or “recombinant” or “synthetic gelatin-like peptide (or polypeptide)”, depending on the method of its production (i.e. by recombinant expression or by chemical synthesis).

It was also found that the lyophilization process can be optimized significantly when the recombinant polypeptide of the invention has no helical structure.

### DESCRIPTION OF THE INVENTION

According to the invention a lyophilized composition is provided comprising as a stabilizer a recombinant or synthetic polypeptide with a calculated glass transition temperature that is higher than about 180 degrees Celsius, comprising at least one stretch of 10 or more consecutive repeats of Gly-Xaa-Yaa triplets and in which at least 20% of the amino acids are present in the form of consecutive Gly-Xaa-Yaa triplets.

The measured glass transition temperature of the composition should also be significantly higher, preferably at least about 5 degrees, more preferably at least about 10 degrees and most preferably 20 degrees Celsius higher, than the measured glass transition temperature of a control composition, which comprises native collagen peptides. “Native collagen” as used herein refers to collagen peptides or polypeptides which were not selected or synthesized to have a high glass transition temperature. In general, native collagen peptides have a calculated Tg of about 170 degrees Celsius or less.

It is noted, that when the Tg of a mixture, composed of a gelatin-like peptide and one or more other compounds, is measured, the measured Tg of the composition may be significantly different from the measured Tg of the substantially pure gelatin-like peptide. For example, the measured Tg of a composition comprising a gelatin-like peptide and sucrose may be significantly lower than the measured Tg of the pure gelatin-like peptide.

Pharmaceutical formulations that are introduced into the bloodstream contain proteins as, for example, a stabiliser, as a drug carrier or as an osmotic colloid. It is long recognized in the art that gelatins are preferred for their low immunogenicity. It is also recognized in the art that recombinant gelatins can advantageously replace gelatins from natural sources to avoid introduction of non-gelatin material. Recently the occurrence of BSE has been a source of concern and a reason to avoid the use of gelatin from natural sources.

Although the use of recombinant gelatins is described for the obvious reasons, and it is suggested that recombinant structures can be optimised there are no teachings as to what such optimisation might comprise.

In our studies on collagen properties we found to our surprise that, although collagen has a repetitive amino acid triplet structure Gly-Xaa-Yaa, wherein a majority of the triplets contain a proline, the glass transition temperature (or Tg) is not uniformly divided over the molecule, and sequences can be selected that have a higher Tg than the average (native) collagen.

The importance of the glass transition temperature is well known in the art of freeze drying or lyophilizing of formulations containing physiologically active substances, like vaccines. In lyophilized formulations one strives for high glass transition temperature. In “Long-Term Stabilization of Biologicals” (Biotechnoloy vol. 12 12 Mar. 1994) F. Franks addresses the importance of high glass transition temperatures in the preservation of biological materials by freeze drying and the desire to further improve the shelf life of such materials. In the formulations for freeze drying, gelatin serves to protect the physiologically active substance whereby the presence of water molecules bound to polar groups of the amino acid residues is thought to be of importance. Residual moisture plays an important role in the shelf life of vaccines. Increased residual moisture levels decrease the glass transition temperature of a lyophilized gelatin/disaccharide composition significantly, resulting in reduced shelf life.

There are many publications on this subject, for example by Phillips et. al in cryobiology 18, 414-419 (1981) or U.S. Pat. No. 801,856. Vaccines like MMR lumps Measles Rubella) have in current formulations a critical Tg, which lies around 47 degrees Celsius under dry conditions but rapidly decreases towards room temperature when small amounts of moisture enter the material. Within one week at 37 degrees Celsius a loss in potency of 50% is reported by M. K. Lala in Indian Pediatrics 2003; 40:311-319 Increasing the Tg even by a few degrees can have a tremendous effect on the shelf life of these vaccines. The problem of a reduced stability of the physiologically active formulations, which are stabilized with gelatins, was solved by the present invention, which is based on the use of new recombinant or synthetic gelatins with an increased Tg in combination with a certain similarity with natural human gelatin amino acid sequences to prevent the occurrence of unwanted immune responses.

A recombinant or synthetic gelatin-like polypeptide according to the invention is preferably a sequence identical to or highly homologous to a native human collagen sequence. To select such an amino acid sequence from a native sequence, “moving Tg averages” (as defined below) are calculated. A sequence is then selected which has a calculated average glass transition temperature of about 10 degrees Celsius higher than the calculated average collagen glass transition temperature of the native starting sequence, preferably about 20 degrees higher, more preferably about 30 degrees higher, even more preferably about 40 degrees higher. This value may differ somewhat between different types of collagen and depend on the presence of propeptides, telopeptides or signal peptides. The average calculated glass transition temperature of native collagen is about 170 degrees Celsius, so that a polypeptide according the invention has a Tg higher than about 180 degrees, preferably higher than about 190 degrees, more preferably higher than about 200 degrees. “About” as used herein refers to a temperature range of 1-4 degrees higher and/or lower than the specified temperature.

Tg increases of less than 10 degrees are also considered, but the effect in the eventual formulation in which disaccharides are present may be reduced to a less significant level.

The calculation method of the glass transition temperature was published by Y. Matveev et al. in Food Hydrocolloids Vol. 11 no. 2 pp. 125-133, 1997. Equations 8 and 9 were used for the actual calculations:

$$\begin{matrix}{T}_{g}^{- 1} = {\sum }_{i = 1}^{20} {ϕ}_{i } {T}_{ g , i}^{- 1} \\ wherein & ( 8 ) \\ {ϕ}_{i} = {n}_{i} Δ {V}_{i} / {\sum }_{i = 1}^{20} {n}_{i} Δ {V}_{i} . & ( 9 )\end{matrix}$$

where the summations i=1 to 20 are the summations of the values for the partial values of T_{g} and ΔV of the separate amino acids given below (V is a measure for the vd Waals volume, as described in Matveev et al. (supra)):

| No. | Amino Acid | T_{g,i} (Kelvin) | ΔVᵢ |
|---|---|---|---|
| 1 | gly | 599 | 47.3 |
| 2 | ala | 621 | 64.4 |
| 3 | val | 931 | 98.6 |
| 4 | leu | 400 | 115.7 |
| 5 | ile | 400 | 115.7 |
| 6 | phe | 528 | 139.9 |
| 7 | pro | 423 | 88.0 |
| 8 | trp | 544 | 196.9 |
| 9 | ser | 311 | 66.1 |
| 10 | thr | 321 | 88.9 |
| 11 | met | 362 | 120.6 |
| 12 | asn | 232 | 94.6 |
| 13 | gln | 312 | 111.7 |
| 14 | cys-SH | 418 | 82.2 |
| 15 | asp | 672 | 80.1 |
| 16 | glu | 487 | 97.2 |
| 17 | tyr | 573 | 136.9 |
| 18 | his | 488 | 118.9 |
| 19 | lys | 258 | 118.1 |
| 20 | arg | 410 | 138.4 |

The model does not appear to take the presence of hydroxyproline into account. However, the correlation with measured values which are presented in the paper of Matveev et al. give a very good correlation between calculated and measured values of gelatin.

For selecting appropriate recombinant or synthetic collagen-like peptides a starting point is for example human Col1A1 (SEQ ID NO: 1), which has a Tg of 163 degrees Celsius calculated from entire sequence.

SEQ ID NO: 1 (human Col1A1):
MFSFVDLRLLLLLAATALLTHGQEEGQVEGQDEDIPPITCVQNGLRYHDRDVW
KPEPCRICVCDNGKVLCDDVICDETKNCPGAEVPEGECCPVCPDGSESPTDQET
TGVEGPKGDTGPRGPRGPAGPPGRDGIPGQPGLPGPPGPPGPPGPPGLGGNFAP
QLSYGYDEKSTGGISVPGPMGPSGPRGLPGPPGAPGPQGFQGPPGEPGEPGASG
PMGPRGPPGPPGKNGDDGEAGKPGRPGERGPPGPQGARGLPGTAGLPGMKGH
RGFSGLDGAKGDAGPAGPKGEPGSPGENGAPGQMGPRGLPGERGRPGAPGPA
GARGNDGATGAAGPPGPTGPAGPPGFPGAVGAKGEAGPQGPRGSEGPQGVRG
EPGPPGPAGAAGPAGNPGADGQPGAKGANGAPGIAGAPGFPGARGPSGPQGPG
GPPGPKGNSGEPGAPGSKGDTGAKGEPGPVGVQGPPGPAGEEGKRGARGEPGP
TGLPGPPGERGGPGSRGFPGADGVAGPKGPAGERGSPGPAGPKGSPGEAGRPG
EAGLPGAKGLTGSPGSPGPDGKTGPPGPAGQDGRPGPPGPPGARGQAGVMGFP
GPKGAAGEPGKAGERGVPGPPGAVGPAGKDGEAGAQGPPGPAGPAGERGEQG
PAGSPGFQGLPGPAGPPGEAGKPGEQGVPGDLGAPGPSGARGERGFPGERGVQ
GPPGPAGPRGANGAPGNDGAKGDAGAPGAPGSQGAPGLQGMPGERGAAGLP
GPKGDRGDAGPKGADGSPGKDGVRGLTGPIGPPGPAGAPGDKGESGPSGPAGP
TGARGAPGDRGEPGPPGPAGFAGPPGADGQPGAKGEPGDAGAKGDAGPPGPA
GPAGPPGPIGNVGAPGAKGARGSAGPPGATGFPGAAGRVGPPGPSGNAGPPGP
PGPAGKEGGKGPRGETGPAGRPGEVGPPGPPGPAGEKGSPGADGPAGAPGTPG
PQGIAGQRGVVGLPGQRGERGFPGLPGPSGEPGKQGPSGASGERGPPGPMGPP
GLAGPPGESGREGAPGAEGSPGRDGSPGAKGDRGETGPAGPPGAPGAPGAPGP
VGPAGKSGDRGETGPAGPAGPVGPAGARGPAGPQGPRGDKGETGEQGDRGIK
GHRGFSGLQGPPGPPGSPGEQGPSGASGPAGPRGPPGSAGAPGKDGLNGLPGPI
GPPGPRGRTGDAGPVGPPGPPGPPGPPGPPSAGFDFSFLPQPPQEKAHDGGRYY
RADDANVVRDRDLBVDTTLKSLSQQIENIRSPEGSRKNPARTCRDLKMCHSDW
KSGEYWIDPNQGCNLDAIKVFCNMETGETCVYPTQPSVAQKNWYISKNPKDK
RHVWFGESMTDGFQFEYGGQGSDPADVAIQLTFLRLMSTEASQNITYHCKNSV
AYMDQQTGNLKKALLLKGSNEIEIRAEGNSRFTYSVTVDGCTSHTGAWGKTVI
EYKTT KTSRLPIIDVAPLDVGAPDQEFGFDVGPVCFL

This Col1A1 sequence still includes the signal sequence (amino acids 1-22) and the amino terminal propeptides (amino acids 23-161 and 1219-1464). The helical collagen sequence is present from amino acid 162 to amino acid 1218. Using a spreadsheet the moving average over a number of amino acids could easily be calculated and displayed. FIGS. 1 to 4 show the result for a moving average of resp. 18, 27, 54 and 81 amino acids. A “moving Tg average” of, for example n=54, means that first the average Tg of the first to the 54^{th} amino acid is calculated, then of the 2^{nd} to 55^{th} amino acid, then from the 3^{rd} to the 56^{th} and so on. These values are then plotted as in FIG. 3, the first datapoint being plotted at the 54^{th} amino acid. The amino acid regions which have a calculated Tg higher than the average calculated Tg of this native collagen (i.e. the average calculated Tg of the complete sequence) can now be identified. It is remarkable that smaller polypeptides allow selection of regions with higher Tg. Calculating a moving average of 54 amino acids allows selection of polypeptide sequences with increased Tg of up to about 200 degrees C. For example a sequence from amino acid 1034 to 1087 of SEQ ID NO: 1 results in a calculated Tg of 208 degrees Celsius. This polypeptide has, thus, a calculated Tg which is 45 degrees Celsius higher than the calculated Tg of the native sequence, which is 163 degrees Celsius calculated for entire sequence. When expressed as such this yields a gelatin-like polypeptide of about 5,000 Dalton. A sequence of about 500 amino acids can be selected from about amino acid 600 to about amino acid 1100 of SEQ ID NO: 1, that still has an average Tg of about 178 degrees Celsius and a molecular weight of about 40,000 to 50,000 Dalton. From about amino acid 590 to 750 of SEQ ID NO: 1 a polypeptide with an average Tg of higher than 180 degrees Celsius can be selected that has a molecular weight of up to about 10,000 to 13,000 Dalton. Polypeptide regions with the desired average Tg such as described here above can be easily calculated also from other collagen sequences, such as Col 1A-2, Col 2A-1, Col 3A-1 and so on. Such collagen sequences are readily available in the art.

When desired, repetitive sequences of these sequences can be expressed to obtain larger molecular weights. Conventional hydrolysed gelatins with a weight of about 3,000 to 15,000 Dalton are applied, preferably between 5,000 and 10,000 Dalton and more preferably between 6,000 and 8,000 Dalton. When desired also larger molecular weights can be obtained by the invention giving a specific advantage for the achievable Tg. Thus in one embodiment the gelatin-like polypeptide has a preferred molecular weight between 3,000 and 15,000 Dalton, more preferably between 5,000 and 10,000, even more preferably between 6,000 and 8,000 Dalton. In another embodiment the gelatin-like polypeptide has a molecular weight between 3,000 and 80,000 Dalton, preferably between 5,000 and 60,000 Dalton, most preferably between 10,000 and 40,000 Dalton.

It was attempted to correlate the Tg of a polypeptide fragment to its structural details. Some correlation was found with the alanine content, as shown in FIG. 5. Although for a moving average of 54 amino acids many of the areas with higher Tg coincide with elevated alanine levels, this correlation is not valid for all regions with a Tg higher than average. Still, with a moving average of 54 amino acids it is likely that a region with higher Tg is found when the polypeptide of 54 amino acids has an alanine content of more than about 1 alanine per 10 amino acids. The presence of bulky amino acid residues can have a negative effect on the Tg of a polypeptide. A correlation was made between the presence of leucine and isoleucine and the Tg over a moving average of 54 amino acids (FIG. 6). In many areas with high Tg, but not all, the concentration of these bulky amino acid residues is low, or they are absent. Bringing valine in the correlation makes it worse, suggesting that valine has less effect on the bulkiness. Considering the sizes of the side chains of the abundantly present prolines it is imaginable that leucine and isoleucine contribute more to the bulkiness than valine. Further, it is desirable that the amount of polar amino acid residues is more than 5% and more preferably more than 7% but less than 15% so that enough water molecules can be bound to protect the lyophilized physiologically active substance.

Gelatin-like recombinant or synthetic polypeptides according to the invention are preferably identical or essentially similar to natural human collagen amino acid sequences, but also non-human sequences (such as rat, rabbit, mouse etc.) can be used, or sequences can be designed that do not occur naturally. The term “essentially similar” means that two peptide sequences, when optimally aligned, such as by the programs GAP or BESTFIT using default parameters, share at least 80 percent sequence identity, preferably at least 90 percent sequence identity, more preferably at least 95 percent sequence identity or more (e.g., 99 or 100 percent sequence identity). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins).

Such sequences would preferably have a high alanine content of more than 10 alanine residues per 100 amino acids, preferably more than 12 per 100 amino acids, more preferably more than 14 per 100 amino acids. Such a designed structure contains polar amino acid residues comparable to natural gelatins. The incorporation of bulky amino acids is to be avoided.

A natural gelatin molecule in its primary amino acid sequence basically consists of repeats of Gly-Xaa-Yaa triplets, thus approximately one third of the total number of amino acids is a glycine. The molecular weight of gelatin is typically large, values of the molecular weight vary from 10,000 to 300,000 daltons. The main fraction of natural gelatin molecules has a molecular weight around 90,000 daltons. The average molecular weight is higher than 90,000 daltons.

Furthermore, characteristic for gelatin is the unusual high content of proline residues. Even more characteristic is that in natural gelatin a number of the proline residues is hydroxylated. Most prominent site of hydroxylation is the 4-position resulting in the presence in the gelatin molecule of the unusual amino acid 4-hydroxyproline. In a triplet 4-hydroxyproline is always found in the Yaa position. Very few proline residues are hydroxylated at the 3 position. In contrast with 4-hydroxyproline, 3-hydroxyproline is always found at the carboxyl side of a glycine residue, thus in the Xaa position in a triplet. Different enzymes are responsible for the formation of 3- or 4-hydroxyproline.

Based on known amino acid compositions, it is estimated that in a gelatin molecule derived from a mammal, approximately 22% of the amino acids are a proline or a hydroxyproline residue. However lower contents of proline and hydroxyproline are found in fish, in particular cold water fish. A rough estimate is that proline and hydroxyproline residues are present in approximately equal amounts, thus in a gelatin molecule derived from a mammal approximately 11% of the amino acids are prolines and approximately 11% are hydroxyprolines. As substantially all hydroxyproline is found in the Yaa position, it is estimated that approximately one third of all triplets in a gelatin molecule comprise a hydroxyproline. The presence of the hydroxyproline residues is responsible for the fact that a gelatin molecule in its secondary structure can adopt a helical conformation.

Furthermore, another amino acid present in natural gelatin that is found in very few other proteins is 5-hydroxylysine. Lysine residues modified in this way are always found in the Yaa position in a triplet.

A predominant feature of gelatins is the presence of Gly-Xaa-Yaa triplets. Such triplets are also present in the gelatin-like proteins of this invention. It is however possible to design a protein in which Gly-Xaa-Yaa triplets or stretches of Gly-Xaa-Yaa triplets are separated by one or more amino acids without significantly altering the gelatin-like character of the protein. Such gelatin-like proteins are comprised by the definition of gelatin-like protein of this invention.

The gelatin-like proteins for use according to the invention can be produced by recombinant methods as disclosed in EP-A-0926543 and EP-A-1014176. For enablement of the production and purification of gelatin-like proteins that can be suitably used in composition according to the invention specific reference is made to the examples in EP-A-0926543 and EP-A-1014176. Thus the gelatin-like proteins can be produced by expression of nucleic acid sequence encoding such polypeptide by a suitable microorganism. The process can suitably be carried out with a fungal cell or a yeast cell. Suitably the host cell is a high expression host cell like Hansenula, Trichoderma, Aspergillus, Penicillium, Neurospora or Pichia. Fungal and yeast cells are preferred to bacteria as they are less susceptible to improper expression of repetitive sequences. Most preferably the host will not have a high level of proteases that attack the collagen structure expressed. In this respect Pichia offers an example of a very suitable expression system. As disclosed in EP-A-0926543 and EP-A-1014176 specifically Pichia pastoris is used as expression system. In one embodiment the micro-organism is also transformed to include a gene for expression of prolyl-4-hydroxylase. In another embodiment the microorganism is free of active post-translational processing mechanism such as in particular hydroxylation of proline.

The selection of a suitable host cell from known industrial enzyme producing fungal host cells specifically yeast cells on the basis of the required parameters described herein rendering the host cell suitable for expression of recombinant gelatin-like proteins suitable in compositions according to the invention in combination with knowledge regarding the host cells and the sequence to be expressed will be possible by a person skilled in the art.

With respect to the design of gelatin-like proteins for use in the invention, several properties of the proteins are addressed. For instance it can be made sure specific amino acids, such as bulky amino acids like leucine or isoleucine which lower the average Tg, will not occur in the protein or only occur infrequently. Otherwise, as discussed above in particular with respect to alanine or polar amino acids, it can be advantageous to introduce a definite number of a specific amino acid in the gelatin-like protein. Yet further the iso-electric point (IEP) can be tuned by the composition of acidic and basic amino acid residues in the gelatin-like proteins.

In one embodiment the composition according to the invention comprises a gelatin-like protein which is homodisperse in nature. Homodisperse means of constant composition and molecular weight. Variations in composition that can occur due to the recombinant production process are allowed. In terms of molecular weight a useful definition of homodispersity would be that at least 90% of the total amount of gelatin-like protein in the composition has a molecular weight that lies within a range of plus or minus 10% around a selected molecular weight. In another embodiment the composition according to the invention comprises two or more gelatin-like proteins each being homodisperse in nature but with different molecular weights (i.e. a bimodal molecular weight distribution). This prevents crystallization during the freeze drying process or during cold storage. The difference in molecular weight results in less probability for crystallization. Preferably the molecular weight difference is between 5000 and 20,000 Dalton, most preferably it is about 10,000 Dalton.

In another embodiment recombinant gelatin-like recombinant or synthetic polypeptides of the invention are free from helical structure. This is achieved by allowing only partial or preferably no hydroxylation of the proline residues. Partial hydroxylation means that less than 10% of the prolines are hydroxylated, preferably less than 5%. The absence of helical structure prevents gelling of the gelatin-like polypeptides, even at low temperatures. This is advantageous in for example vaccine formulations which are dissolved in water before injection. The dissolved vaccine can now be used without the necessity to heat it to prevent gelling.

Non gelling gelatin-like polypeptides are also advantageously used in the freeze drying process. In freeze drying of gelatin, the solution is first frozen before the actual freeze drying is started. This process is described in for example U.S. Pat. No. 3,892,876. It is important that the gelatin is frozen in the sol state and not in the gel-state, because otherwise the lyophilized gelatin will not dissolve again after freeze drying. Recombinant gelatin-like proteins of the invention make it possible to freeze dry more concentrated gelatin solutions, resulting in a higher amount of vaccine in the same time, a 10-20% shorter freeze drying time, reducing damage to the physiologically active substance or the gelatin during freeze drying and reducing freeze drying costs.

The starting point for the gelatin-like protein for use in the invention can also be an isolated gene encoding a naturally occurring gelatin molecule, which is processed further by recombinant means. Preferably the gelatin-like protein used according to the invention resembles a human native amino acid sequence with this difference that in essence hydroxyproline residues are absent.

When produced by recombinant means, especially by expression of recombinant genes in yeasts, the proteins for use according to the invention preferably do not contain a combination of methionine and arginine in 1-4 position (Met-Xay-Xaz-Arg), as such a sequence is sensitive to enzymatic proteolysis.

It may be noted that the proteins for use according to the invention can also be partly or wholly produced by methods other than DNA expression, e.g. by chemical protein synthesis.

In order to obtain the composition of the invention one or more gelatin-like proteins of the invention are mixed with the physiologically active compound. As an aid in vitrification a saccharide can be added. Preferably this is a disaccharide like sucrose. Depending on the application also a variety of other compounds can be added like amino acids, other proteins than gelatin, etc.

The composition of the invention comprises an amount of gelatin-like proteins which usually lies in the range from 2-60 weight %.

### DESCRIPTION OF THE FIGURES

FIG. 1: Tg of a moving average of n=18 for human COL1A1

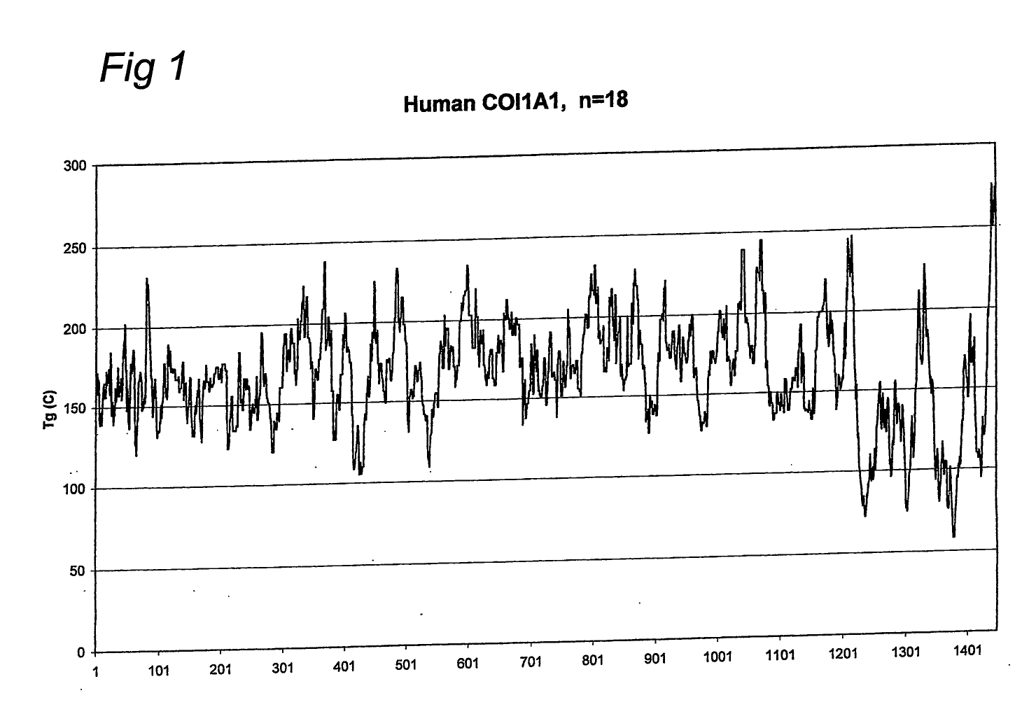

FIG. 2: Tg of a moving average of n=27 for human COL1A1

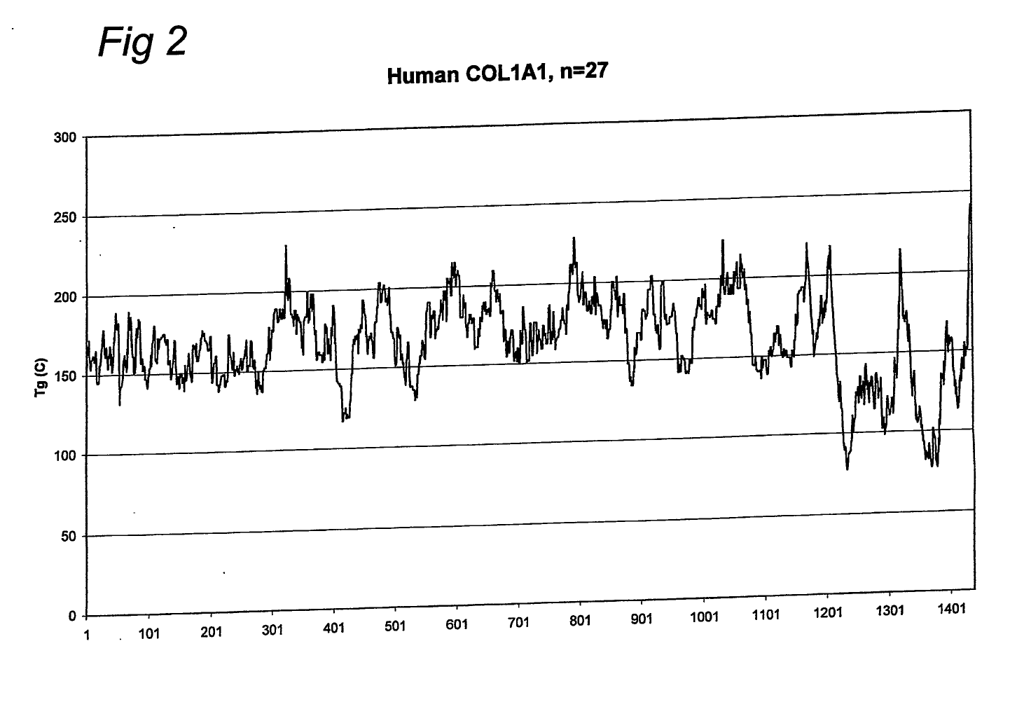

FIG. 3: Tg of a moving average of n=54 for human COL1A1

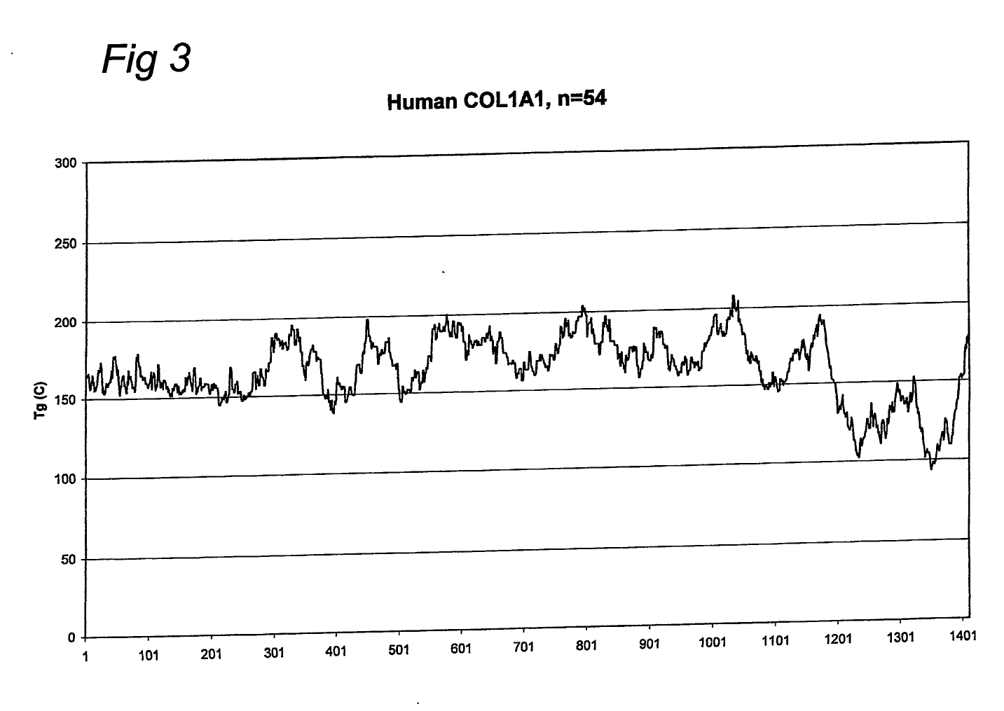

FIG. 4: Tg of a moving average of n=81 for human COL1A1

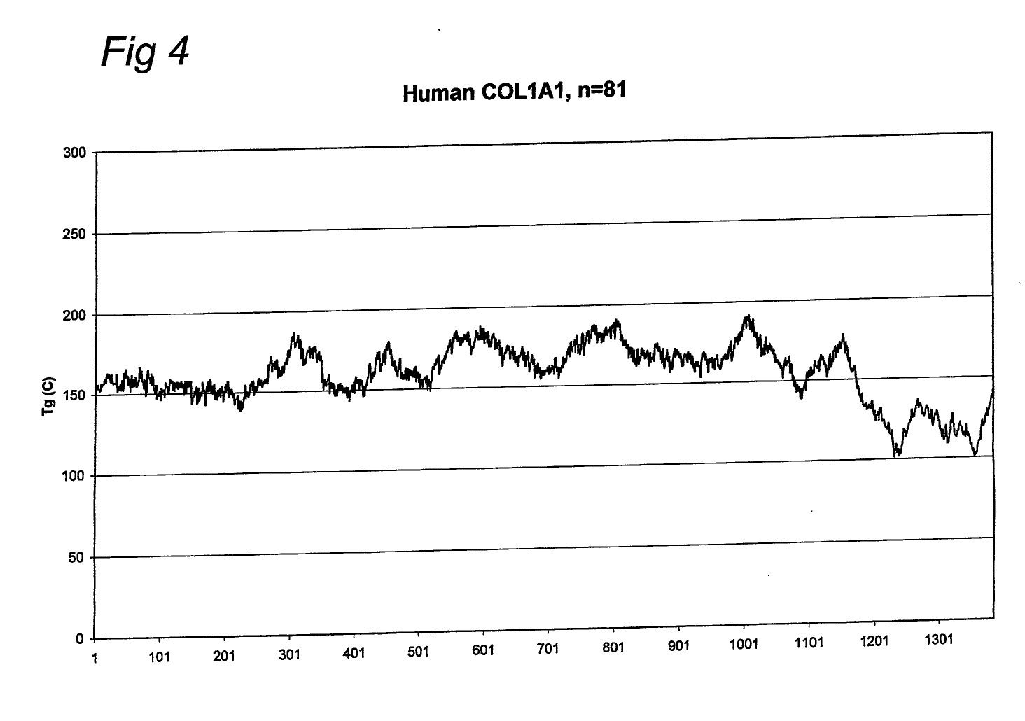

FIG. 5: Tg of a moving average of n=54 for human COL1A1; correlation with alanine content

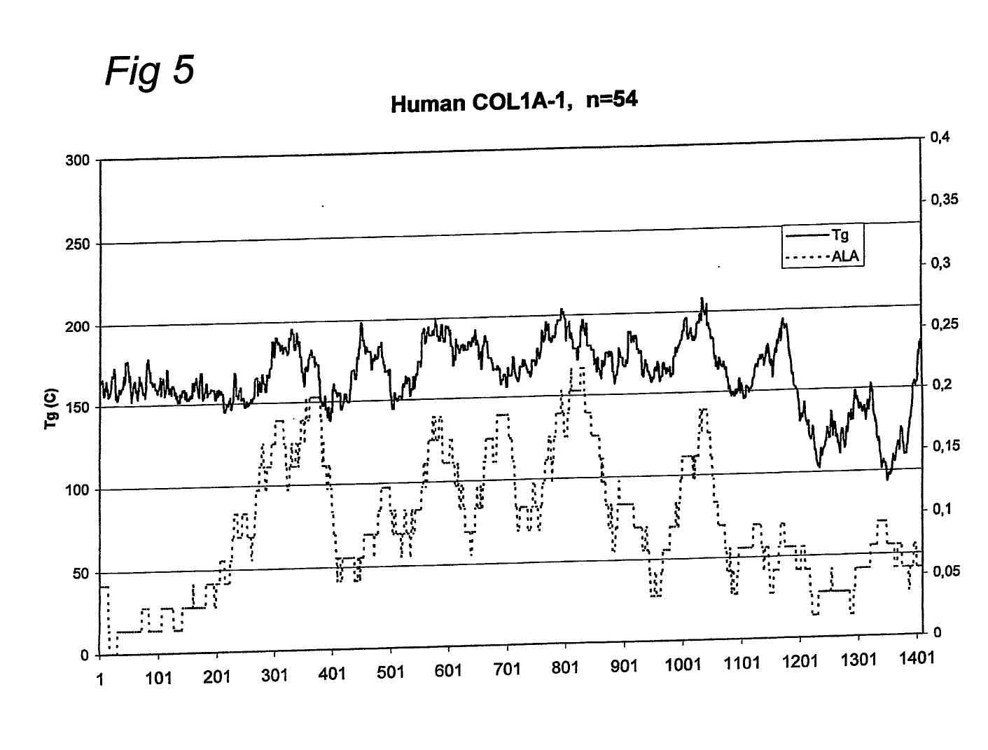

FIG. 6: Tg of a moving average of n=54 for human COL1A1; correlation with leucine+isoleucine content

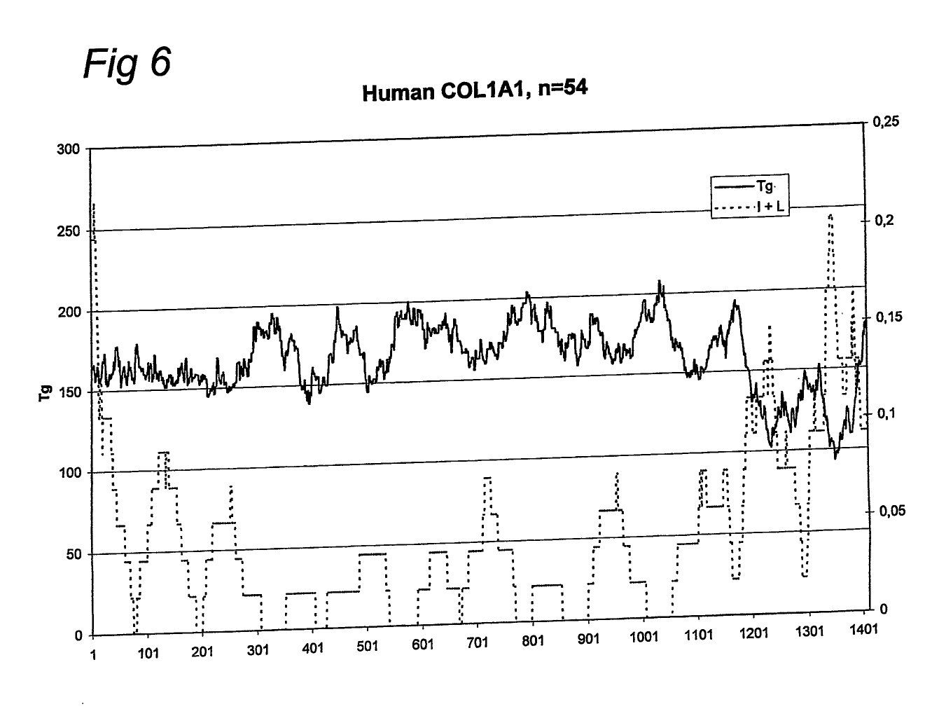

### EXAMPLES

### Example 1

### Recombinant Gelatin-Like Peptide

A gelatin with an increased glass transition temperature was produced by starting with the nucleic acid sequence that encodes for a part of the gelatin amino acid sequence of human COL1A1-1. The methods as disclosed in EP-A-0926543, EP-A-1014176 and WO01/34646 were used. The sequence of this gelatin according to the invention is given below (SEQ ID NO: 2):

GDRGETGPAGPPGAPGAPGAPGPVGPAGKSGDRGETGPAGPAGPVGP
AGARGPA (amino acid 1034 to 1087 of SEQ ID NO: 1)

Molecular weight: 4590 Da, isoelectric point pI=6.2

This sequence was selected from the total COL1A1-1 sequence (SEQ ID NO: 1) by the method as described in this invention. A glass transition temperature of 208 degrees Celsius was calculated for this selected sequence. The average glass transition temperature of total COL1A1-1 (SEQ ID NO: 1) is 163 degrees Celsius. Therefore the calculated gain in glass transition temperature is 45 degrees Celsius.

### Example 2

### Measurement of Glass Transition Temperature

The recombinant gelatin as described in example 1 was mixed with sucrose in a ratio of 60/40 wt % gelatin/sucrose, which is typical for MMR vaccine. An aqueous solution of 10% was made of this mixture. This solution was quickly frozen in liquid nitrogen and subsequently it was freeze dried for 48 hours at −55 degrees Celsius. The freeze dried sample was further dried in a vacuum exsiccator with silicagel.

DSC (Differential Scanning Calorimetry) was done using a Perkin Elmer DSC 7 instrument under nitrogen atmosphere (flow 20 ml/min). The applied temperature program was:
1 minute hold at 60 degrees Celsius
60 to 230 degrees Celsius at a heating rate of 5 degrees per minute

The glass transition temperature was determined according to the half Cp extrapolated method.

Residual moisture amounts were determined by TGA (Thermo Gravimetric Analysis) using a Perkin Ebmer TGA 7 under nitrogen atmosphere (flow 20 ml/min).

The applied temperature program was:
25-60 degrees Celsius with a heating rate of 5 degrees per minute
1 minute hold at 60 degrees Celsius

60 to 300 degrees Celsius at a heating rate of 5 degrees per minute

Residual moisture amount of the dry recombinant gelatin/sucrose mixture was found to be in the range of 1-2 wt %.

The glass transition temperature of the dry recombinant gelatin/sucrose mixture was measured to be 130 degrees.

As a reference the glass transition temperature of native COL1A1 in the same mixture with sucrose was found to be 116 degrees.

The measured Tg of the mixture comprising the selected recombinant gelatin was thus 14 degrees Celsius higher than the analogous mixture comprising the (non-selected) native gelatin, showing that selection of gelatin-like peptides with a higher calculated Tg also result in mixtures comprising such peptides having a higher measured Tg.

1. A lyophilized composition comprising a physiologically active substance and a stabilizer, characterized in that the stabilizer is a recombinant or synthetic gelatin-like polypeptide comprising at least one stretch of 10 or more consecutive repeats of Gly-Xaa-Yaa triplets and in which at least 20% of the amino acids are present in the form of consecutive Gly-Xaa-Yaa triplets and wherein said recombinant polypeptide has a calculated glass transition temperature of higher than 180 degrees Celsius.

2. A composition as in claim 1 wherein said recombinant or synthetic gelatin-like polypeptide has a molecular weight between 3,000 Dalton and 80,000 Dalton preferably between 5,000 Dalton and 60,000 Dalton and more preferably between 10,000 and 40,000 Dalton.

3. A composition as in claim 1 wherein said recombinant or synthetic gelatin-like polypeptide has a molecular weight between 3,000 Dalton and 15,000 Dalton preferably between 5,000 Dalton and 10,000 Dalton and more preferably between 6,000 and 8,000 Dalton.

4. A composition as in claim 1 wherein the glass transition temperature of the recombinant or synthetic gelatin-like polypeptide is higher than 190 degrees Celsius preferably higher than 200 degrees Celsius.

5. A composition as in claim 1 wherein the recombinant or synthetic gelatin-like polypeptide has a bimodal molecular weight distribution.

6. A composition as in claim 1 wherein the recombinant or synthetic gelatin-like polypeptide is free from helical structure.

7. A composition as in claim 1 wherein the number of hydroxyproline residues in the recombinant or synthetic gelatin-like polypeptide is less than 5% of the total number of amino acid residues preferably less than 2%.

8. A recombinant or synthetic gelatin-like polypeptide comprising at least one stretch of 10 or more consecutive repeats of Gly-Xaa-Yaa triplets and in which at least 20% of the amino acids are present in the form of consecutive Gly-Xaa-Yaa triplets and wherein said recombinant gelatin-like polypeptide has a calculated glass transition temperature of higher than 180 degrees Celsius.

9. Process for lyophilizing compositions comprising a physiological active substance and a stabilizer characterized in that the stabilizer is a recombinant or synthetic gelatin-like polypeptide comprising at least one stretch of 10 or more consecutive repeats of Gly-Xaa-Yaa triplets and in which at least 20% of the amino acids are present in the form of consecutive Gly-Xaa-Yaa triplets and less than 5% of the total number of amino acid residues are hydroxyproline residues and wherein said recombinant gelatin-like polypeptide has a calculated glass transition temperature of higher than 180 degrees Celsius.

10. A composition as in claim 2 wherein the glass transition temperature of the recombinant or synthetic gelatin-like polypeptide is higher than 190 degrees Celsius preferably higher than 200 degrees Celsius.

11. A composition as in claim 3 wherein the glass transition temperature of the recombinant or synthetic gelatin-like polypeptide is higher than 190 degrees Celsius preferably higher than 200 degrees Celsius.

12. A composition as in claim 2 wherein the recombinant or synthetic gelatin-like polypeptide has a bimodal molecular weight distribution.

13. A composition as in claim 3 wherein the recombinant or synthetic gelatin-like polypeptide has a bimodal molecular weight distribution.

14. A composition as in claim 4 wherein the recombinant or synthetic gelatin-like polypeptide has a bimodal molecular weight distribution.

15. A composition as in claim 2 wherein the recombinant or synthetic gelatin-like polypeptide is free from helical structure.

16. A composition as in claim 3 wherein the recombinant or synthetic gelatin-like polypeptide is free from helical structure.

17. A composition as in claim 4 wherein the recombinant or synthetic gelatin-like polypeptide is free from helical structure.

18. A composition as in claim 5 wherein the recombinant or synthetic gelatin-like polypeptide is free from helical structure.

19. A composition as in claim 2 wherein the number of hydroxyproline residues in the recombinant or synthetic gelatin-like polypeptide is less than 5% of the total number of amino acid residues preferably less than 2%.

20. A composition as in claim 3 wherein the number of hydroxyproline residues in the recombinant or synthetic gelatin-like polypeptide is less than 5% of the total number of amino acid residues preferably less than 2%.
